(19) United States
(12) Patent Application Publication
(10) Pub. No.: US 2009/0253929 A1
(43) Pub. Date: Oct. 8, 2009

(54) Chemical Compounds

(75) Inventors: Allison Noble, Leicestershire (GB); Matthew Perry, Leicestershire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/721,290

(22) PCT Filed: Dec. 7, 2005
(86) PCT No.: PCT/SE05/01864

§ 371 Date: Jun. 8, 2007

(30) Foreign Application Priority Data
Dec. 9, 2004 (SE) 0403003-7

(51) Int. Cl. C07C 25/18; C07C 255/50; C07C 17/26

(52) U.S. Cl. 558/425; 570/129; 570/171

(57) ABSTRACT

The present invention concerns a compound of formula (I): wherein R¹ is C₁₋₄ alkyl, or benzyl optionally substituted by halogen, C₁₋₄alkyl (optionally substituted by halogen or C₁₋₄ alkoxy), C₁₋₄ alkoxy, C₁₋₄ alkoxycarbonyl, nitro or cyano; and a process for preparing a compound of formula (I).

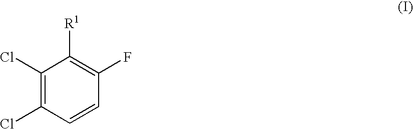

The present invention concerns 3-alkyl-1,2-dichloro-4-fluorobenzene compounds and a process for their preparation.

3-Alkyl-1,2-dichloro-4-fluorobenzene compounds are useful in the preparation of modulators (for example antagonists) of CCR3 chemokine receptor activity. For example 3-alkyl-1,2-dichloro-4-fluorobenzene compounds are useful pharmaceutical intermediates in the preparation of [(phenoxy)-[1,4′]bipiperidinyl-1′-yl]-(phenyl)-methanone derivatives (see, for example, WO03/004487, WO2004/099144 and WO2004/087659).

Thus, the present invention provides a compound of formula (I):

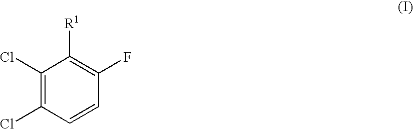

wherein R¹ is C₁₋₄ alkyl, or benzyl optionally substituted by halogen, C₁₋₄ alkyl (optionally substituted by halogen or C₁₋₄ alkoxy), C₁₋₄ alkoxy, C₁₋₄ alkoxycarbonyl, nitro or cyano.

Halogen is, for example, fluoro or chloro.

Alkyl is straight or branched chain and is, for example, methyl, ethyl, iso-propyl, n-butyl, sec-butyl or tert-butyl.

Alkoxy is straight or branched chain and is, for example, methoxy or ethoxy.

In one aspect the invention provides a compound of formula (I) wherein R¹ is C₁₋₄ alkyl or benzyl. In another aspect the present invention provides a compound of formula (1) wherein R¹ is C₁₋₄ alkyl (for example ethyl or methyl). In yet another aspect the present invention provides a compound of formula (1) wherein R¹ is methyl.

In a further aspect the present invention provides a process for preparing a compound of formula (I), the process comprising:
a. reacting a compound of formula (II):

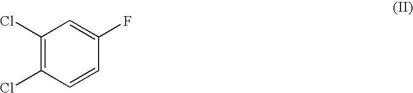

with a suitable strong base at a temperature in the range −80 to −30° C. in a suitable solvent to form a carbanion of a compound of formula (II); and,
b. reacting the carbanion of a compound of formula (II) with a compound R¹L,
   wherein L is a suitable leaving group, in a suitable solvent, and at a temperature in the range −80 to −10° C. (for example −50 to −30° C.).

Suitable strong bases are, for example, C₁₋₁₀ alkyl (for example C₁₋₆ alkyl, such as C₄) lithiums (such as n-butyl lithium, sec-butyl lithium), a di-C₁₋₁₀ alkyl (for example di-C₁₋₆ alkyl) lithium amide base (such as lithium diiso-propylamide), an aryl lithium (such as a phenyl lithium) or an arylalkyl lithium (such as a benzyl lithium).

In a still further aspect of the invention a suitable strong base is, for example a C₁₋₆ alkyl (for example C₄) lithium (such as n-butyl lithium, sec-butyl lithium) or a di-C₁₋₆ alkyl lithium amide base (such as lithium diiso-propylamide). In another aspect of the invention the strong base is n-butyl lithium.

A suitable solvent for steps a and b of the process is an ether (for example tetrahydrofuran [THF], methyl tert-butyl ether or dioxan).

The leaving group L is, for example, halogen (such as bromine or iodine), triflate or methylsulfate. In a further aspect of the invention L is methylsulfate.

In another aspect the present invention provides a process as hereinbefore described wherein, in step b, the carbanion of a compound of formula (II) is added to the compound R¹L.

In yet another aspect the present invention provides a process as hereinbefore described whereinbetween 1 and 1.5 molar equivalents of strong base is used {such as between 1.1 and 1.25 equivalents (for example between 1.15 and 1.2 equivalents) of strong base}.

In a further aspect the present invention provides a process as hereinbefore described wherein an excess of compound R¹L over strong base is used.

In yet another aspect the present invention provides a process as hereinbefore described wherein, in step b, the compound R¹L is added to the carbanion of a compound of formula (II).

In a further aspect the present invention provides a process as hereinbefore described wherein steps a and b are carried out at a temperature in the range −60 to −10° C. (for example −60 to −30° C.).

The invention will now be illustrated by the following non-limiting Examples. In the Examples the following apply, unless stated otherwise:
(i) when given, ¹H NMR data is quoted and is in the form of delta values for major diagnostic protons, given in parts per million (ppm) relative to tetramethylsilane (TMS) as an internal standard, determined at 300 MHz or 400 MHz using perdeuterio DMSO-D6 (CD₃SOCD₃) or CDCl₃ as the solvent unless otherwise stated;
(ii) Reverse phase analytical HPLC was run on a Hewlett Packard Series 1100 using Waters “Symmetry” C8 column 3.5 μn; 4.6×50 mm column. The gradient was either:
   “Standard” (0.1% Ammonium acetate/Acetonitrile 75% to 5% in 3 minutes, 2 mL/minutes), or,
   “Fast” (0.1% Ammonium acetate/Acetonitrile 45% to 5% in 2.5 minutes; 2 mL/minutes); and,
(iii) mass spectra (MS) were run with an electron energy of 70 electron volts in the chemical ionisation (CI) mode using a direct exposure probe; where values for m/z are given, generally only ions which indicate the parent mass are reported, and unless otherwise stated the mass ion quoted is the positive mass ion −(M+H)⁺.

### EXAMPLE 1

This Example illustrates the preparation of 1,2-dichloro-4-fluoro-3-methylbenzene.

n-Butyl lithium (75 ml, 2.48 M in hexanes, Aldrich, 1.1 equivalents) is added over 1 h by syringe pump to a solution of 1,2-dichloro-4-fluorobenzene (20 ml, 28.1 g, 1 equivalents) in anhydrous THF (180 ml) at −40° C., keeping the temperature between −45° C. and 40° C. The solution is stirred at −40° C. for 15-30 minutes and then a solution of dimethylsulfate (19 ml, 1.2 equivalents) in THF (20 ml) is added over 1 h by syringe pump, keeping temperature between −45° C. and −40° C. The mixture is then stirred at −40° C. for 15-30 minutes and then allowed to warm to room temperature. To the solution is then added 5% w/v (or 5% w/w) brine (60 ml) followed by aqueous ammonia solution (0.88 NH₃, 40 ml). The mixture is stirred vigorously for 30 minutes and then the layers separated. The bottom layer is discarded and the top layer washed with 5% w/v brine solution (100 ml) by again stirring for 30 minutes and then separating the layers. The top layer is then analysed for product by GC, evaporated to dryness and if necessary purified by distillation.

Gas Chromatography Analysis showed 87.1% 1,2-dichloro-4-fluoro-3-methylbenzene, 2.1% of a dimethylated fluorodichlorobenzene and 8.0% of a second dimethylated fluorodichlorobenzene.

NMR δ_{CDCl3}: 2.35 (d, 3H, J=2.4 Hz), 6.92 (t, 1H, J=8.8 Hz), 7.27 (ddq, 1H, J=8.8, 5.4, 0.6 Hz) ppm.

MS (GC-MS) M⁺ (EI)=178.

### EXAMPLE 2

This Example illustrates the preparation of 1,2-dichloro-4-fluoro-3-methylbenzene.

1,2-dichloro-4-fluorobenzene (30 mL) and N,N,N′,N′-tetramethylethane-1,2-diamine (45 mL) in THF (400 mL) were cooled to −78° C. sec-Butyl lithium (1.3M, 315 mL) was added dropwise over 2 h. The resultant mixture was stirred at −78° C. for 4 h. Iodomethane (18.2 mL) was added. The reaction mixture was allowed to warm to room temperature overnight. Saturated NH₄Cl solution was added and the mixture was extracted using ether and ethyl acetate, the organic layers were combined, washed with 2M HCl solution, dried over MgSO₄ and the solvents were removed. The residue was distilled under reduced pressure (water aspirator, ca 20 mmHg) to give the title compound (17.3 g; b.pt. 75° C. at water aspirator, approximately 20 mmHg).

HPLC (standard gradient) retention time 2.63 minutes.

### EXAMPLE 3

This Example illustrates the preparation of 1,2-dichloro-4-fluoro-3-methylbenzene

1,2-Dichloro-4-fluorobenzene (0.25 mL) was dissolved in THF (2 mL) and the solution was cooled to −78° C. n-Butyl lithium (2.5M in hexanes, 1.0 mL) was added dropwise and the resulting solution was stirred at −78° C. for 15 minutes then allowed to warm to −40° C. The solution was held at −40° C. for 15 minutes then recooled to −78° C. and methyl triflate (0.30 mL) was added dropwise. The resulting solution was allowed to warm slowly to ambient temperature. Aqueous ammonium chloride was added and the reaction mixture was extracted twice with ether. The organic phases were combined and washed with brine, dried, filtered and concentrated. HPLC of the residue shows the major component to be the title compound (Retention time standard gradient 2.58, identical to a previously prepared sample of title compound).

### EXAMPLE 4

This Example illustrates the preparation of 1,2-dichloro-3-ethyl-4-fluorobenzene.

1,2-Dichloro-4-fluorobenzene (1.3 ml) was dissolved in THF (10 ml) and the resultant solution was cooled to −78° C. n-Butyl lithium (10M, 1.2 ml) was added dropwise over 5 minutes. The resultant solution was stirred at −78° C. for 5 minutes then allowed to warm to ca −40° C. and held at this temperature for 15 minutes. The solution was cooled to −78° C. and then iodoethane (1.24 ml) was added. The resultant solution was allowed to warm to 10° C. pH7 Buffer was added followed by ethyl acetate and diethyl ether. The phases were separated and the aqueous phase was extracted twice with diethyl ether. The organics were combined, washed with brine, dried, filtered and concentrated to give the title compound, contaminated with diethyl ether and ethyl acetate (2.37 g).

GCMS 97.75% retention time 4.61 minutes (M⁺ (EI) 192/194/196; bp 177) (Agilent 6890/5973 GC/MSD HP5-MS column, 30 m×0.25 mm with a film thickness of 0.25 um, 90-310° C. at 30° C./minutes).

¹H NMR δ_{(CDCl3)} 1.18 (3H, t), 2.84 (2H, qd), 6.92 (1H, t), 7.27 (1H, dd).

The following compounds were prepared analogously using appropriate alkylating agents:

### EXAMPLE 5

### 2-Benzyl-3,4-dichloro-1-fluorobenzene using benzyl bromide

HPLC (fast) retention time 1.94.

¹ H NMR δ_{(CDCl3)} 4.19 (2H, d), 6.98 (1H, t), 7.16-7.36 (6H, m).

### EXAMPLE 6

### Methyl 3-(2,3-dichloro-6-fluorobenzyl)benzoate using 3-bromomethyl-benzoic acid methyl ester

HPLC (fast) retention time 1.77.

¹H NMR δ(CDCl3) 3.90 (3H, s), 4.23 (2H, d), 6.97-7.04 (1H, m), 7.30-7.42 (3H, m), 7.88 (1H, d), 7.93 (1H, s).

### EXAMPLE 7

This Example illustrates the preparation of 1,2-dichloro-4-fluoro-3-methylbenzene.

n-Butyl lithium (33.9 kg, 2.7M in hexanes, Aldrich, 1.17 equivalents) was added over 35 minutes to a solution of 1,2-dichloro-4-fluorobenzene (17.95 kg, 1 equivalents) in anhydrous THF (105 L) at −45° C., keeping the temperature between −45° C. and −40° C. The solution was then cooled to −55° C. and added to a solution of dimethylsulfate (16.4 kg, 1.2 equivalents) in THF (96 kg) at −19° C. over 30 minutes, temperature −15° C. The mixture was then warmed to 20.5° C. To the solution was then added a mixture of sodium chloride (1.9 kg) and ammonium hydroxide solution (22 kg, 28%) as a solution in water (36 L) at 21° C. The mixture was stirred vigorously for 30 minutes at 20-25° C. and then the layers separated. The bottom layer was discarded and the top layer washed with a solution of sodium chloride (3.3 kg) in water (60 L) by stirring for 30 minutes at 20-25° C. and then separating the layers. The lower layer was discarded and the solvent removed from the top layer by distillation at 40° C. and 60 mbar. The residue was then purified by distillation at 90-100° C. and 11-10 mbar to give the 1,2-dichloro-4-fluoro-3-methylbenzene (14.8 kg, 76%) as a colourless, waxy solid. (GC: 0.1% starting fluorodichlorobenzene, 93.18% product, 0.2% ethylated fluorodichlorobenzene, 1.57% first dimethylated fluorodichlorobenzene, 4.48% second dimethylated fluorodichlorobenzene.)

The product was redistilled together with a second batch at 102-105° C. and 15.5-13 mbar to give the title compound (29.01 kg, 78%) as a colourless waxy solid. (GC 0.55% starting fluorodichlorobenzene, 96.99% title compound, 0.22% ethylated fluorodichlorobenzene, 0.6% first dimethylated fluorodichlorobenzene, 1.64% second dimethylated fluorodichlorobenzene.)

NMR δ_{CDCl3}: 2.35 (d, 3H, J=2.4 Hz), 6.92 (t, 1H, J=8.8 Hz), 7.27 (ddq, 1H, J=8.8, 5.4, 0.6 Hz).

MS (GC-MS) M⁺ (EI)=178

### EXAMPLE 8

This Example illustrates the preparation of 1,2-dichloro-4-fluoro-3-methylbenzene

Lithium di-iso-propylamide (11.3 ml, 1.8M, 1.2 equivalents) was added over 10 minutes to a solution of 1,2-dichloro-4-fluorobenzene (2 ml, 2.8 g, 1 equivalents) in anhydrous THF (20ml) at −60° C., keeping the temperature below −60° C. The solution was stirred at −60° C. for 20 minutes, warmed to −40° C., stirred at −40° C. for 30 minutes and then cooled to −60° C. A solution of dimethylsulfate (2.25 ml, 1.4 equivalents) in THF (2 ml) was added dropwise over 20 minutes, keeping temperature between −60° C. and −55° C. The mixture was stirred at −60° C. for 30minutes, warmed to room temperature overnight and then quenched with saturated brine solution (10 ml). Diethylether (10 ml) was added, followed by aqueous ammonia solution (0.88 NH₃, 20 ml). The mixture was stirred for 30 minutes at room temperature and the top layer analysed for product by GC. (GC: 81.6% title compound, 2.1% first dimethylated fluorodichlorobenzene, 11.9% second dimethylated fluorodichlorobenzene.)

1. A compound of formula (I): 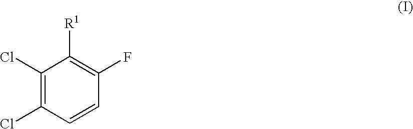
wherein R¹ is C₁₋₄ alkyl, or benzyl optionally substituted by halogen, C₁₋₄ alkyl (optionally substituted by halogen or C₁₋₄ alkoxy), C₁₋₄ alkoxy, C₁₋₄ alkoxycarbonyl, nitro or cyano.

2. A compound of formula (I) as claimed in claim 1 wherein R¹ is C₁₋₄ alkyl.

3. A compound of formula (I) as claimed in claim 1 or 2 wherein R¹ is methyl.

4. A process for the preparation of a compound of formula (I), the process comprising:
a. reacting a compound of formula (II): 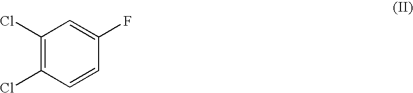
with a suitable strong base at a temperature in the range −80 to −30° C. in a suitable solvent to form a carbanion of compound of formula (II); and,
b. reacting the carbanion of compound of formula (II) with a compound R¹L, wherein L is a suitable leaving group, in a suitable solvent, and at a temperature in the range −80 to −30° C.

5. A process as claimed in claim 4 wherein the strong base is a C₁₋₁₀ alkyl lithium or a di-C₁₋₁₀ alkyl lithium amide base.

6. A process as claimed in claim 4 or 5 wherein the strong base is n-butyl lithium.

7. A process as claimed in claim 4, 5 or 6 wherein L is halogen, triflate or methylsulfate.

8. A process as claimed in claim 4, 5, 6 or 7 wherein steps a and b are carried out at a temperature in the range −60 to −30° C.

9. A process as claimed in claim 4, 5, 6, 7 or 8 wherein, in step b, the carbanion of a compound of formula (II) is added to the compound R¹L.

10. A process as claimed in claim 4, 5, 6, 7, 8 or 9 wherein the suitable solvent for steps a and b is an ether.
